(19) United States
(12) Patent Application Publication
(10) Pub. No.: US 2006/0105325 A1
(43) Pub. Date: May 18, 2006

(54) Adsorbent with enhanced protein binding capacity and selectivity

(76) Inventors: Richard C. Willson, Houston, TX (US); Sindhu Balan, Pearland, TX (US); Ajish S.R. Potty, Houston, TX (US)

Correspondence Address: Benjamin Aaron Adler;ADLER & ASSOCIATES, 8011 Candle Lane, Houston, TX, 77071, US

(21) Appl. No.: 11/273,562

(22) Filed: Nov. 14, 2005

Related U.S. Application Data
(60) Provisional application No. 60/627,712, filed on Nov. 12, 2004.

(51) Int. Cl. C12Q 1/70; C12Q 1/68

(52) U.S. Cl. 435/5; 435/6

(57) ABSTRACT

The invention discloses an adsorbent composition in which each ligand has a cluster of functional groups. This adsorbent composition is designed to enhace binding affinity and specificity for biomolecules. The invention also discloses a method for adsorbing a biomolecule onto the disclosed adsorbent composition. A biomolecule can be adsorbed to the disclosed composition either for qualitative analysis or purification of the biomolecule. The invention also discloses a kit for qualitative analysis or recovery of a biomolecule.

### CROSS-REFERENCE TO RELATED APPLICATION

This non-provisional application claims benefit of provisional U.S. Ser. No. 60/624,603, filed Nov. 12, 2004, now abandoned.

### FEDERAL FUNDING LEGEND

This invention was produced in part using funds obtained through a grant from the National Science Foundation (Grant No. CTS-0004544). Consequently, the federal government has certain rights in this invention.

### BACKGROUND OF THE INVENTION

1. Field of the Invention

The present invention relates generally to the field of chromatographic separation of biomolecules. More specifically, the present invention relates to enhancing capacity and selectivity of ion exchange adsorbents by forming small charge clusters on the adsorbent matrix.

2. Description of the Related Art

Ion exchange chromatography is one of the most widely used processes for purification of a wide range of biomolecules (1,2). The high binding capacity of ion exchange adsorbents, excellent product recovery, use of aqueous buffer systems, and easy scale up are some of the factors that favor ion exchange chromatography. The process is versatile in that a single adsorbent can be used to purify an array of biomolecules. On the other hand, this versatility makes the process less selective than a technique like affinity chromatography.

Efforts to improve ion exchange adsorbents have primarily focused on mass transfer characteristics and on increasing overall ligand density through the use of tentacular adsorbents (3,4,5) and of polyions, such as polyethyleneimine, polysulfonic acid and polylysine (6,7). Adsorbent capacity can be greatly improved by increasing ligand density. However, a concurrent increase in selectivity cannot be achieved by increasing just the ligand density for an adsorbent. Recovery of a molecule often calls for a fine balance between capacity and selectivity of downstream processing steps to ensure high yield and purity.

A probable approach to improve selectivity in ion exchange is by looking at the charge distribution patterns of a group of similar biomolecules. Focusing on proteins, it is clear that many of them have a patch or sequence of amino acids that is conserved across species (8,9). If the physiological role of the protein involves electrostatic interactions, this conserved amino acid sequence is made up of acidic or basic amino acids (10,11,12). Mediating multivalent contact with an ion exchange adsorbent through these high charge density patches can improve not only selectivity for a family of proteins, but also capacity of the adsorbent for these proteins. Earlier work using site directed mutation alluded to the presence of such charged clusters in proteins that is ‘ion exchange active’ or preferred by an adsorbent (13,14).

Traditional ion exchangers with dispersed charge distributions display a heterogeneous landscape of adsorption sites due to variations in local charge density. Electrolytes used in ion exchange chromatography decrease the range of electrostatic interactions between the protein and ion exchanger (the Debye length at 100 mM of a 1:1 electrolyte, such as NaCl, is 0.96 nm), limiting the degree of “averaging” over local variations and heightening the effects of surface charge heterogeneity (15). These effects also limit the opportunity for selectivity based on the affinity advantage of clusters of concentrated charge seen in protein functional domains.

Proteins also can have zones that are rich in hydrophobic amino acids or amino acids with sulfhydryl, i.e., —SH groups. An adsorbent which can take advantage of such zones by presenting concentrated clusters of functional groups that can mediate hydrophobic or thiophillic interactions may probably show enhanced binding capacity and selectivity for these proteins, as compared to traditional hydrophobic or thiophillic adsorbents. Likewise, if nucleic acids are considered, they have clusters of negative charges and clusters of aromatic bases. Thus, adsorbents that have positively charged clusters or chelated metal clusters can interact selectively with nucleic acids via the cluster of negative charges and the cluster of aromatic bases, respectively.

From the above discussion it is clear that the distribution pattern of interacting functional groups on an adsorbent matrix may be critical in defining selectivity of adsorbents. By enhancing selectivity of adsorbents the number of steps required to purify a molecule can be reduced. This not only improves product yield, but also makes the product recovery process more economical.

The prior art is deficient in the lack of adsorbents with small clusters of functional groups for enhanced binding capacity and selectivity for purifying molecules. The present invention fulfills this long-standing need in the art.

### SUMMARY OF THE INVENTION

The present invention is directed to an adsorbent composition. The composition comprises a support and a ligand associated with this support. The ligand in this composition comprises a cluster of functional groups such that the majority of clusters contain substantially the same number of functional groups.

The present invention is directed to a related adsorbent composition comprising a support material and a peptide ligand. The peptide ligand comprises about 3 to about 10 amino acid molecules or derivatives thereof.

The present invention also is directed to a method of adsorbing a biomolecule to the disclosed adsorbent composition by contacting the biomolecule with the adsorbent composition. The biomolecule may be adsorbed either for qualitative analysis or for its recovery and purification.

The present invention is directed further to a kit comprising the disclosed adsorbent compositions. The kit further comprises accessories required to enable adsorption of a biomolecule.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. The above may be better understood by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIG. 1 illustrates the binding of a cationic protein to a dispersed charges and clustered charges adsorbent.

FIG. 2 shows the adsorption isotherms of cytochrome c on dispersed charge (E) and clustered charge (E5) agarose at 25° C. in 10 mM Tris, pH 8 at 26 mM NaCl where the carboxyl group density is 5.7 μmole/mL of gel. The ligand in each case is coupled to CNBr activated agarose.

FIG. 3 shows the adsorption isotherms of lysozyme on dispersed charge (E) and clustered charge (E5) agarose at 25° C. in 10 mM Tris, pH 8 at 26 mM NaCl where the carboxyl group density is 5.7 μmole/mL of gel. The ligand in each case is coupled to CNBr activated agarose.

FIG. 4 shows the adsorption isotherms of cytochrome c on dispersed charge (E) and clustered (E5) charge agarose at 25° C. in 10 mM Tris, pH 8 at 26 mM NaCl where the carboxyl group density is 22 μmole/mL of gel. The ligand in each case is coupled to CNBr activated agarose.

FIG. 5 shows the adsorption isotherms of lysozyme on dispersed charge (E) and clustered charge (E5) agarose at 25° C. in 10 mM Tris, pH 8 at 26 mM NaCl where the carboxyl group density is 22 μmole/mL of gel. The ligand in each case is coupled to CNBr activated agarose.

FIG. 6 shows the Hill plots of cytochrome c on E5-agarose at varying salt concentrations where X is the free protein concentration at equilibrium and Y is the concentration of bound protein. The ligand in each case is coupled to CNBr activated agarose.

FIG. 7 shows the Hill plots of lysozyme on E5-agarose at varying salt concentrations where X is the free protein concentration at equilibrium and Y is the concentration of bound protein. The ligand in each case is coupled to CNBr activated agarose.

FIG. 8 shows the Z plots for cytochrome c on E-agarose and E5 agarose at salt concentrations ranging from 26-75 mM NaCl. The ligand in each case is coupled to CNBr activated agarose.

FIG. 9 shows the Z plots for lysozyme on E-agarose and E5 agarose at salt concentrations ranging from 26-75 mM NaCl. The ligand in each case is coupled to CNBr activated agarose.

FIG. 10 shows the adsorption isotherms of α-lactalbumin (Ca²⁺ saturated) on lysinamide and pentalysinamide agarose at 25° C. in 10 mM Tris, pH 8 at 26 mM NaCl where the amino group density is 5.0 μmole/mL of gel. The ligand in each case is coupled to CNBr activated agarose.

FIG. 11 shows the adsorption isotherms of α-lactalbumin (Ca²⁺ depleted) on lysinamide and clustered pentalysinamide agarose at 25° C. in 10 mM Tris, pH 8 at 26 mM NaCl where the amino group density is 5.0 μmole/mL of gel. The ligand in each case is coupled to CNBr activated agarose.

FIG. 12 shows the adsorption isotherms of cytochrome b₅ (wild type) on dispersed lysinamide and clustered pentalysinamide agarose at 25° C. in 10 mM Tris, pH 8 at 26 mM NaCl where the amino group density is 5.0 μmole/mL of gel. The ligand in each case is coupled to CNBr activated agarose.

FIG. 13 show the adsorption isotherms of cytochrome b₅ (E11Q) on dispersed lysinamide and clustered pentalysinamide agarose at 25° C. in 10 mM Tris, pH 8 at 26 mM NaCl where the amino group density is 5.0 μmole/mL of gel. The ligand in each case is coupled to CNBr activated agarose.

FIG. 14 shows the adsorption isotherm of cytochrome b₅ (E44Q) on dispersed lysinamide and clustered pentalysinamide agarose at 25° C. in 10 mM Tris, pH 8 at 26 mM NaCl where the amino group density is 5.0 mmole/mL of gel. The ligand in each case is coupled to CNBr activated agarose.

FIG. 15 shows the adsorption isotherms of cytochrome c on dispersed charge (E) and clustered charge (E5) agarose at 25° C. in 10 mM Tris, pH 8 at 26 mM NaCl where the carboxyl group density is 5.7 μmole/mL of gel. The ligand in each case is coupled to aldehyde activated agarose.

FIG. 16 shows the adsorption isotherms of lysozyme on dispersed charge (E) and clustered charge (E5) agarose at 25° C. in 10 mM Tris, pH 8 at 26 mM NaCl where the carboxyl group density is 5.7 μmole/mL of gel. The ligand in each case is coupled to aldehyde activated agarose.

### DETAILED DESCRIPTION OF THE INVENTION

In one embodiment the instant invention provides an adsorbent composition comprising a support material and a ligand. Each ligand comprises a cluster of functional groups. The number of functional groups per cluster is the same for the majority of ligands. The functional groups are selected such that the adsorbent composition can be used for ionic, hydrophobic, thiophillic or metal affinity interactions with a biomolecule. The functional groups can for example be carboxylate moieties, amine moieties, hydrophobic moieties, chelating moieties, a sulfopropyl group, a diethylaminoethyl group, a sulphydryl group or a carboxymethyl group.

Proteins often show clusters of amino acids that are essential for the physiological role supported by proteins. The amino acid clusters maybe ionic thiophillic or hydrophobic depending on cofactors and ligands that the protein may have to bind in order to elicit a physiological response. Generally, such clusters comprise a small number of amino acids. These concentrated ionic, thiophillic or hydrophobic patches can allow for selective binding of proteins to an adsorbent. The disclosed adsorbent composition is directed towards such selective binding of proteins and other biomolecules. The ligand of the disclosed adsorbent composition provides a cluster of functional groups which can bind an interacting cluster on the biomolecule. To increase selectivity it is preferable that these clusters comprise a small number of functional groups. If the number of functional groups per cluster is very high, then such an adsorbent will randomly bind all biomolecules in which interacting functional groups are dispersed over a wide area of the biomolecule and not be present as concentrated clusters. Accordingly it is preferable that the ligand of the disclosed composition comprises about 3 to about 10 functional groups.

The ligands that can be used to prepare the disclosed adsorbent composition include peptides, oligonucleotides, sugar polymers, or fatty acids. These ligands have clusters of functional groups, such as clusters of positively charged amino groups or negatively charged carboxyl groups, that are present in a peptide. Ligands with clusters of functional groups also can be synthesized by polymerizing monomers that have functional groups required for adsorbing a biomolecule.

The support material for the disclosed adsorbent composition can be, but not limited to, agarose, alginate, polystyrene, cellulose, polyacrylamide, dextran, polyethylene glycol, fullerenes, glass, graphite or a combination thereof. Different chemistries for coupling ligands either directly or via a crosslinker to these materials are well known in the art. The support material can be porous, magnetic or weighted for rapid settling. Rapid settling of adsorbent is important both in batch and column mode of adsorption. The support material can also be in the form of a gel, a glass slide, one or more fibers, a microchip, a paper, or beads.

In a related embodiment the instant invention further provides a peptide composition comprising a support material and a peptide ligand associated with this support material. The peptide ligand in this composition consists of about 3 to about 10 amino acid molecules or derivatives thereof. The peptide can comprise anionic amino acids, such as glutamic acid and aspartic acid, cationic amino acids, such as lysine and arginine, thiophillic amino acids, such as cysteine, and hydrophobic amino acids, such as tyrosine and tryptophan. Common derivatives of amino acids, such as amides, also can be used to prepare the peptide ligand. The support material of the instant composition can be agarose. The peptide ligand can be coupled to agarose using different chemistries that are well known in the art.

In another embodiment the instant invention provides a method for adsorbing a biomolecule onto the disclosed adsorbent composition comprising the step of contacting a biomolecule with the instant adsorbent composition. Adsorption of a biomolecule to the instant adsorbent composition is either for qualitative analysis or recovery and purification of the biomolecule. The invention discloses that an adsorbent prepared by clustering positive charges was able to identify small perturbations in the anionic protein α-lactalbumin that disturbed a negative cluster of aspartic amino acids in this protein. This demonstrates the analytical potential of the instant adsorbent composition.

The method of adsorbing a biomolecule to the instant adsorbent composition may be carried out in a mode such as fast liquid chromatography, expanded bed chromatography, simulated bed chromatography, electrophoresis and capillary electrophoresis. Biomolecules, such as a protein, a nucleic acid, an oligonucleotide, a virus or a cell organelle, can be recovered and purified using this method. Nucleic acids can be thought of as clusters of aromatic bases. If the adsorbent composition of the instant invention has clusters of chelated metal, then it will selectively bind a nucleic acid fragment that is in a configuration which exposes a cluster of aromatic bases. For example in double stranded DNA and plasmids the aromatic bases owing to base stacking interactions are buried inside the molecule and hence not available for interaction with chelated metal ligands. However, in linear RNA and oligonucleotides these bases are exposed and hence can bind chelated metal ligands. Accordingly, it is contemplated that, depending on the cluster of aromatic bases that can interact with a cluster of chelated metal, different RNA species can be separated or purified.

The adsorbent compositions disclosed herein can be used in a system that is used to separate biomolecules. Examples of some such systems are chromatography systems and electrophoresis sytems. It is contemplated that such systems can be used to separate biomolecules for qualitative analysis or for recovery and purification of the biomolecule.

Table 1 shows the extensions and preferred values of major parameters.

TABLE 1
Extensions and preferred values of major parameters

| Parameter | Variants |
|---|---|
| Ligands | anionic, cationic, affinity (metal), thiophillic |
| Clusters | Scatter, dense, homogeneous, heterogeneous |
| Support | beads, membrane, magnetic beads, gels, fiber, paper, |
| | microchip |
| Chemistry of | cross linked agarose, cross linked dextran, |
| support | polyacrylamide, silica, alginate, polystyrene, |
| | polyethylene glycol, fullerenes, graphite, glass |
| Ligand immobi- | Avidin/biotin, amine, carbodimide, thiol, gold/thiol, |
| lization | metal chelate affinity, aldehyde, mixed-ligand, |
| chemistry | adsorptive, covalent |
| Operation mode | Linear chromatography, displacement |
| | chromatography, expanded bed chromatography, |
| | radial chromatography, simulated moving bed |
| | chromatography, batch adsorption |
| Biomolecules to | proteins, peptides, nucleic acid, oligonucleotides, |
| be separated/ | drugs, spores, fungus, yeast, mold, artificial RNA, |
| purified | insulin, pesticide, enzyme substrate, enzyme reaction |
| | product, virus, yeast artificial chromosomes, bacterial |
| | artificial chromosomes, anthrax spore, bacteria |
| Source from | Blood sample, air filtarte, tissue biopsy, cancer cell, |
| which a | soli sample, water sample, whole prganism, forensic |
| biomolecule is | samples, livestock, Pcr products, cell cultures, |
| to be purified | microbial colonies, vascular plaques, transplant tissue |
| Sample prepa- | Acids, bases, detergents, phenol, ethanol, |
| ration agents | isopropanol, chaotropes, enzymes, protease, |
| prior to | nuclease, polymerase, adsorbent, ligase, primer, |
| adsorption | nucleotide, filters, hydroxyapatite, silica, zirconia |
| Sample prepa- | Filteration, centrifugation, electrophoresis, PCR, |
| ration methods | precipitation, cell culturing |
| prior to | |
| adsorption | |
| Utility of the | Pathogen discovery, biodefense, research, adulterant |
| adsorbents | detection, environmental monitoring, law enforcement, |
| | food safety, taxonomic classification, microbial |
| | ecology |

In yet another embodiment the instant invention provides a kit comprising the adsorbent compositions disclosed herein. This kit also may comprise a column, a glass slide, a microchip, buffers or a combination thereof to enable adsorbtion of a biomolecule. This kit can be used for qualitative analysis or recovery and purification of a biomolecule.

As used herein a cluster refers to a macromolecule which has at least about 3 to about 10 functional groups. For example pentaglutamic acid has a cluster of 6 carboxylate groups, pentalysinamide has a cluster of 5 ε-amino groups and a pentanucleotide has a cluster of 5 aromatic bases. A macromolecule may also be synthesized by polymerizing monomers such that each monomer contributes at least one functional group.

The following examples are given for the purpose of illustrating various embodiments of the invention and are not meant to limit the present invention in any fashion.

### EXAMPLE 1

Clustered Charge and Dispersed Charge Cation Exchange Adsorbents Using CNBR-Activated Agarose

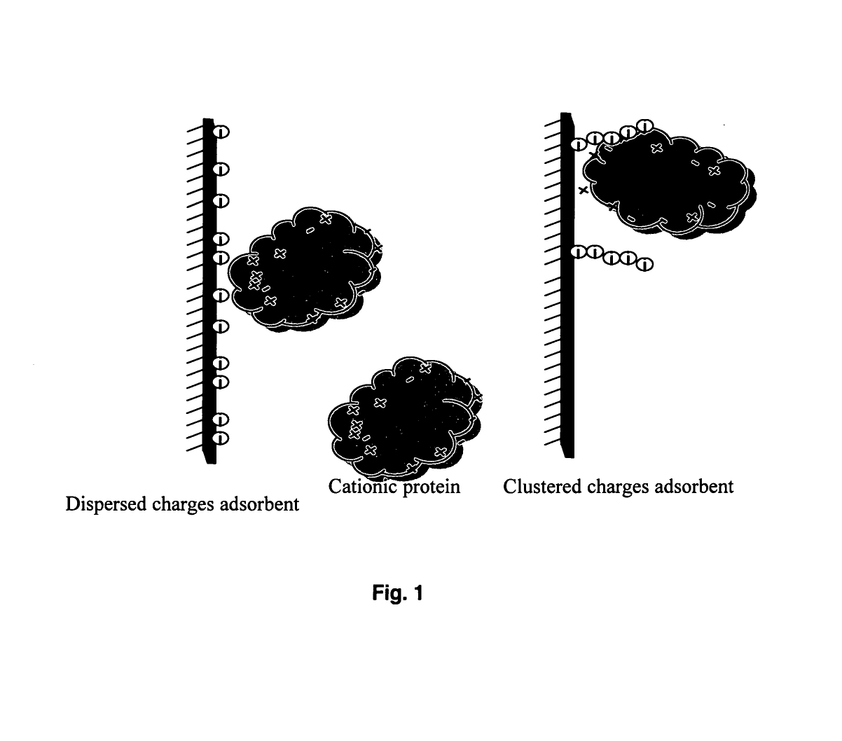

1 gram of CNBr-activated agarose was washed with 250 mL of 1 mM HCl to remove additives added during lyophyllization and suspended in 8 mL of binding buffer (0.5 M NaHCO₃, pH 8.3 containing 0.5 M NaCl) in a 15 mL Falcon tube. 1 gram of the gel swells to give ˜3.5 mL of gel. Glutamic acid or pentaglutamic acid was dissolved in the same buffer and a volume chosen to give the desired ligand density was added to the gel suspension. For example, for the low-density cation exchange adsorbents, 2.85 μmole glutamic acid (E) or 0.95 μmole pentaglutamic acid (E5) was added per mL of the gel respectively, as each glutamic acid contributes two carboxylate groups and each pentaglutamic acid contributes six carboxylate groups. After ligand addition, the reaction tube was placed on a gyratory shaker at 25° C. for 1 hr. The suspension was then filtered and the supernatant analyzed for amino acid content by ninhydrin assay and peptide content using bicinchoninic acid assay. The gel was washed with three volumes of binding buffer and then suspended and shaken 2 hr in 12 mL of 0.1 M Tris-HCl buffer, pH 8 containing 0.5 M of NaCl, to quench any active sites still present. This was followed by filtering the suspension and washing the gel alternately with 0.5 M NaCl in acidic 0.1 M sodium acetate buffer, pH 5.0 and basic 0.1 M Tris-HCl buffer, pH 8.0 (total wash volume was 5 gel volumes). Finally the gel was suspended in 10 mM Tris-HCl buffer, pH 8.0, which is also the binding buffer used for adsorption isotherms. FIG. 1 illustrates a dispersed charges and a clustered charges adsorbent including protein binding thereto.

### EXAMPLE 2

Adsorption Isotherms of Cationic Proteins

Adsorbent (25 μl, E or E5-agarose) was aliquoted into a set of 1.5 mL Eppendorf micro centrifuge tubes. To these tubes varying amounts of 10 mM Tris-HCl, pH 8.0, 2 M NaCl and protein solution (lysozyme or cytochrome c; concentration ranging from 50-1600 μg) was added to achieve a final volume of 1 mL in each of the tubes. The tubes were then rotated on a gyratory shaker at 25° C. for 1 hr. After 10 min centrifugation at 16000×g, protein in the supernatant was quantified at 280/300 nm in a Beckman Coulter DU 530 spectrophotometer. To each of the pellets, 1 mL of binding buffer was added, and the sample vortexed and centrifuged to wash away unbound protein from the interstitial space. The tubes were centrifuged again and the supernatant discarded. The protein was then eluted with 1 mL of 10 mM Tris-HCl, pH 8.0+1 M NaCl. The tubes were rotated on a gyratory shaker at 25° C. for 1 hr and then recentrifuged. The supernatant was analyzed spectrophotometrically at 280/300 for bound protein content.

These cation exchangers were tested with hen egg white lysozyme (molecular weight 14.3 KDa) and bovine heart cytochrome c (molecular weight 12.2 KDa). These proteins both have a net positive charge at pH 8 [16,17], and their similar sizes preclude any major differences in their adsorption behavior arising from steric or transport effects.

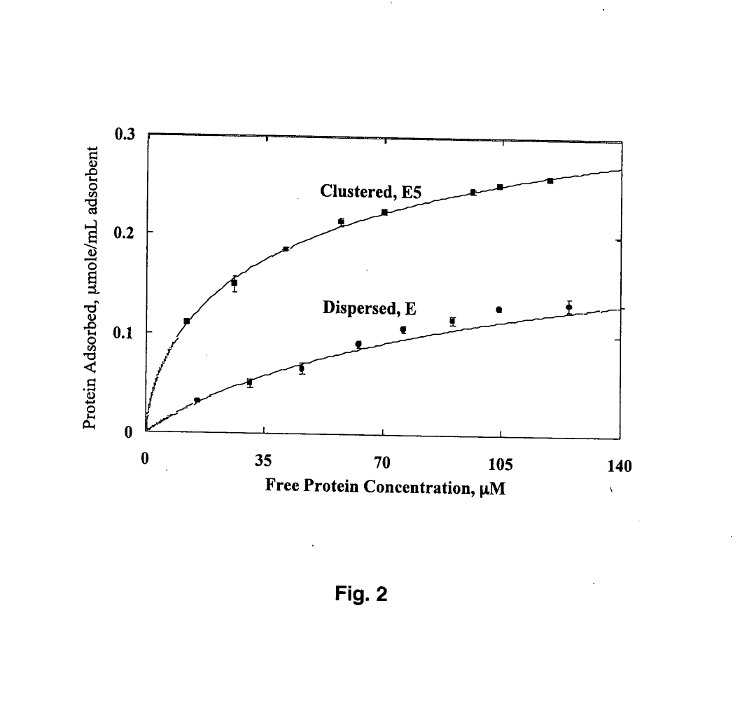

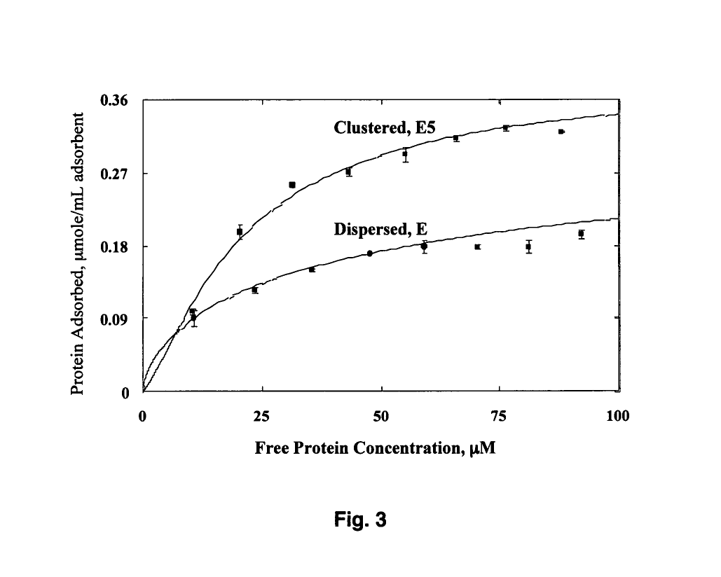

Adsorption isotherms for cytochrome c (FIG. 2) on the lower density E and E5 adsorbents shows a marked increase in apparent affinity for the clustered charge adsorbent as compared to the dispersed charge adsorbent of the same charge density. The apparent affinity Qₘₐₓ/K_{d}ⁿ^{_{H}} increases 18-fold and the apparent capacity (Qₘₐₓ) is higher by a factor of 1.8 for the clustered charge adsorbent under these experimental conditions. FIG. 3, by contrast, illustrates that while clustering increases capacity, the initial affinity of lysozyme adsorption is similar for the clustered and the dispersed adsorbents.

The enhancement of initial apparent binding affinity and apparent capacity of E5-agarose is significantly higher for cytochrome c as compared to lysozyme. This is consistent with the presence of two positively-charged patches on the cytochrome c surface, which are spatially segregated from the acidic amino acid residues (18,19). The general enhancement in binding by clustered adsorbents can be attributed to the increased fraction of adsorbent charges incorporated in clusters of “effective” total charge and charge density. When the protein in question has an inherent charge cluster, this binding is further enhanced as the cluster can interact favorably with the clustered charges on the adsorbent. Jennisen has demonstrated the requirement for a “critical” hydrophobic density to retain proteins in hydrophobic interaction and it is possible that the performance of clustered ion exchange adsorbents is enhanced by related mechanisms (20).

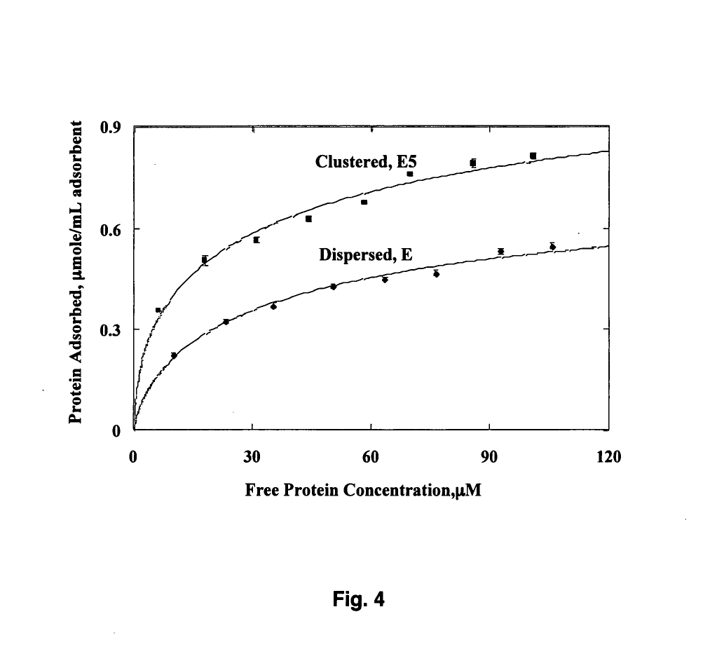

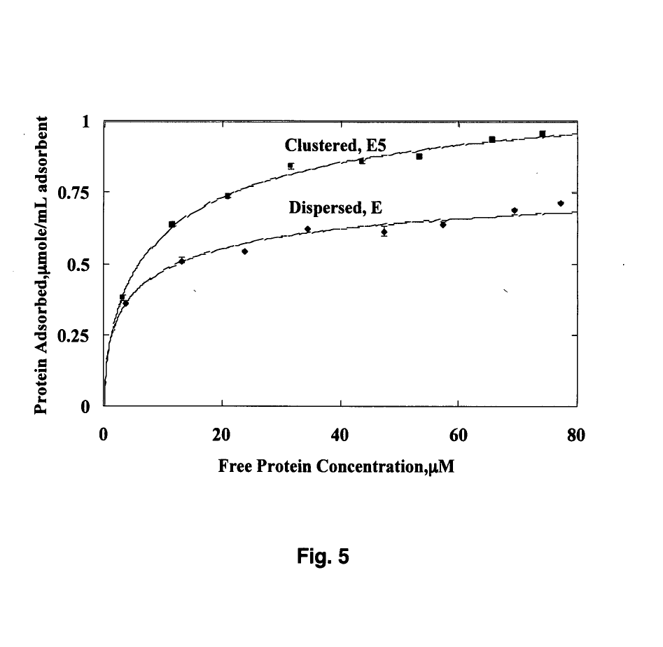

Even at high carboxylate density, clustered E5-agarose showed higher initial affinity for cytochrome c as compared to E-agarose (FIG. 4). The selective enhancement in binding capacity for cytochrome c as compared to lysozyme for the clustered adsorbent is evident (FIGS. 4-5). The enhancement of adsorption due to clustering is greater for the low-density clustered charge adsorbent for both cytochrome c and lysozyme. As the ligand density increases, the probability of stochastic formation of adsorbent charge clusters exceeding any given “critical” charge density or number of charges within a given radius increases. This may explain the greater clustering-induced enhancement of initial affinity (18-fold) for cytochrome c at lower ligand density as compared to 3.3-fold obtained at higher ligand density.

### EXAMPLE 3

Hill and Z Plots for Cytochrome c and Lysozyme

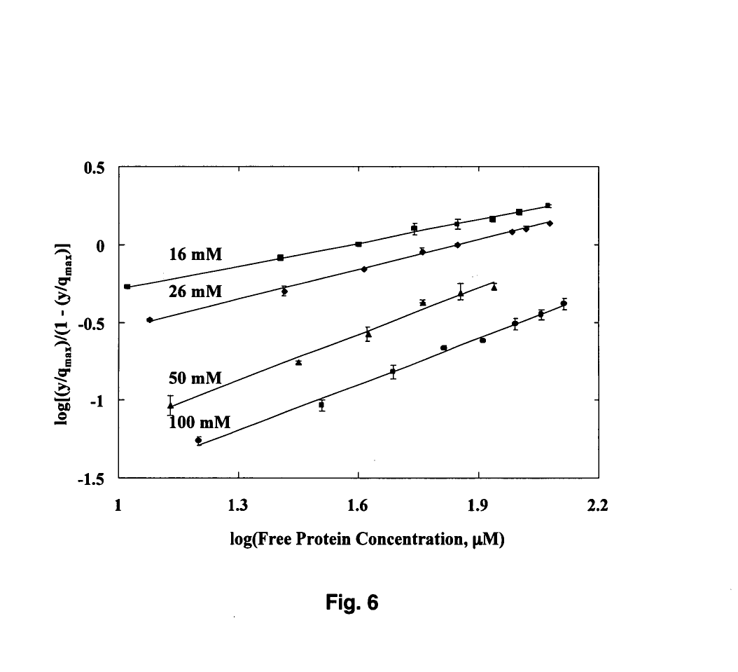

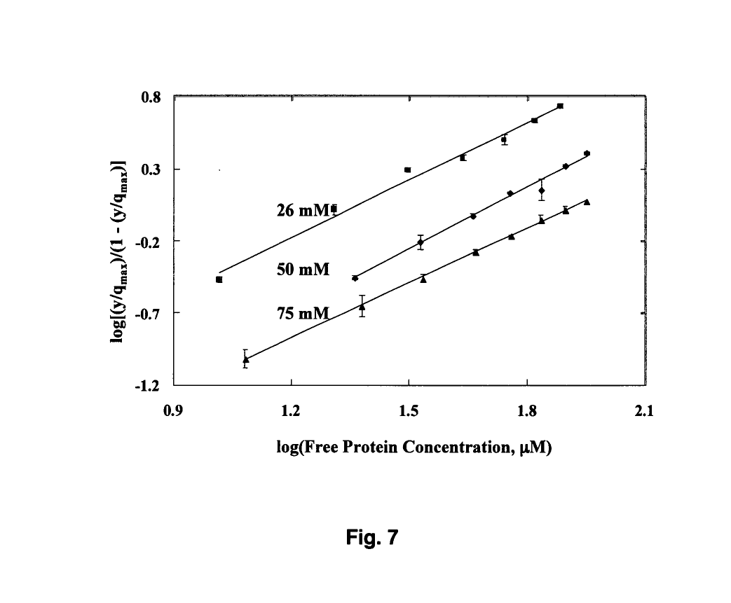

A common Qₘₐₓ value was chosen for the lower-density adsorbents across all salt concentrations for a given adsorbent-protein combination to obtain the Hill plots. As shown in FIGS. 6 and 7, all curves were well fit using the highest observed value of Qₘₐₓ. At values ±10% of the chosen Qₘₐₓ value there was no statistically significant change in the quality of the isotherm fits. The other Hill parameters (n_{H} and K_{d}) showed a corresponding maximum variation of ±10% but the trends in their numerical values remained unchanged. At low salt concentrations, the heterogeneity index, n_{H}, ranged from 0.5 to 0.7 for lysozyme and cytochrome c, with both set of adsorbents (Table 1). On increasing the salt concentration, the value of n_{H} was closer to unity for all protein adsorbent combinations. This decrease in binding heterogeneity may be a result of salt-mediated shielding of low affinity interactions (21). For lysozyme some positive cooperativity effect was seen with E5 (n_{H} values >1). This may be attributed to the known self-association behavior of lysozyme molecules at higher bound protein concentration adsorbed on E5-agarose. The dissociation constant, K_{d}, for E5-agarose was consistently lower compared to E-agarose for both cytochrome c and lysozyme. As expected, K_{d} increased with salt concentration (Table 2).

TABLE 2
Hill constants K_{d} and n_{H} as a function of NaCl concentration for
cytochrome c and lysozyme at 25° C. in 10 mM Tris, pH 8.0.

| | NaCl | | | | |
|---|---|---|---|---|---|
| | Concentration | E-agarose | | E5-agarose | |
| Protein | mM | K_{d}, mM | n_{H} | K_{d}, mM | n_{H} |
| Cytochrome c | 6 | 63.8 ± 2.10 | 0.63 ± 0.03 | — | — |
| | 16 | — | — | 38.2 ± 0.76 | 0.49 ± 0.01 |
| | 26 | 125 ± 19.30 | 0.94 ± 0.02 | 70.9 ± 1.23 | 0.63 ± 0.01 |
| | 50 | 176 ± 12.36 | 1.10 ± 0.04 | — | — |
| | 100 | — | — | 329 ± 20.50 | 1.00 ± 0.03 |
| Lysozyme | 26 | 51.3 ± 1.04 | 0.76 ± 0.01 | 20.1 ± 0.70 | 1.19 ± 0.05 |
| | 50 | 82.6 ± 2.36 | 1.00 ± 0.03 | 64.2 ± 1.08 | 1.19 ± 0.03 |
| | 75 | — | — | 76.7 ± 0.66 | 1.27 ± 0.01 |
| | 100 | 146.8 ± 4.52 | 1.17 ± 0.03 | — | — |

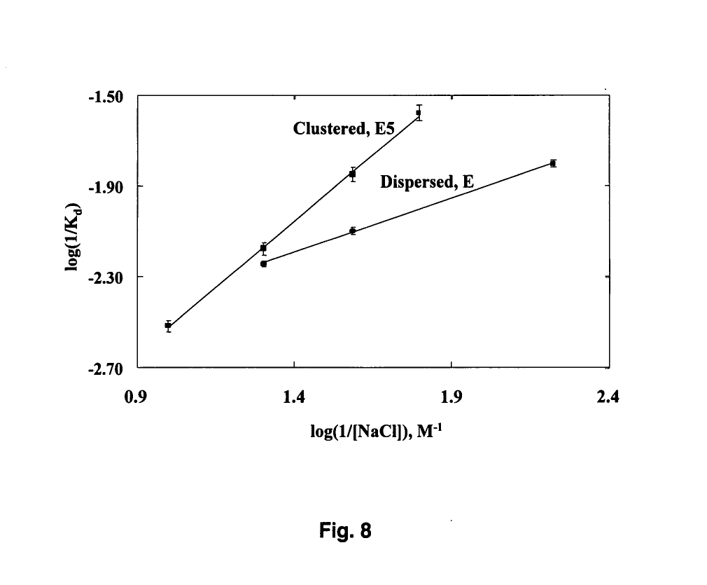

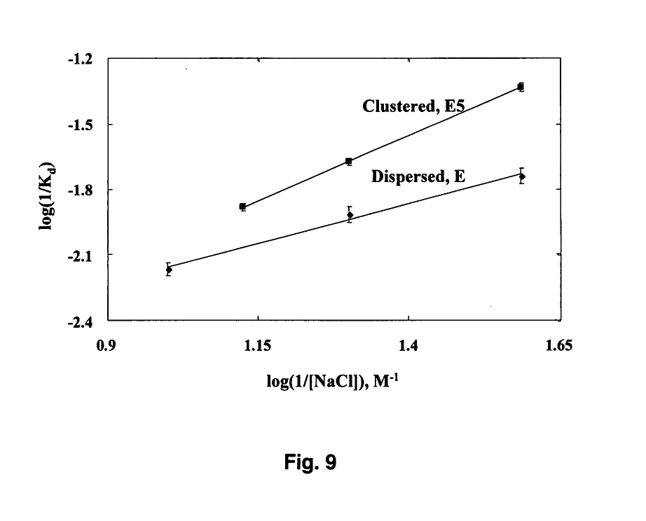

The Z plots for cytochrome c and lysozyme on E-agarose and E5-agarose (FIGS. 8-9) show a linear dependence of the Hill binding constant, K, (the intercept of the corresponding Hill plot) on reciprocal salt concentration as predicted by the stoichiometric displacement model (22,23). For NaCl, the slope of this plot gives the apparent number of interacting sites, Z, on the protein surfaces. As protein molecules can adsorb in different orientations and each orientation can have a different number of (fractional) interactions with the adsorbent surface, Z can have fractional values (24).

The Z values obtained from FIGS. 8 and 9 are tabulated in Table 3. For lysozyme, a slight decrease in the Z values is observed with E5-agarose, and this could be due to the enhanced positive cooperativity with the clustered adsorbent. With n_{H} values >1, the binding of the first protein increases the affinity for the second protein thereby reducing the average number of interacting sites. Also, sigmoidal isotherms are observed with n_{H} values >1, which have lower initial binding affinity compared to Langmuir isotherms (n_{H}=1) with the same parameters Qₘₐₓ and K_{d}. For cytochrome c, however, the apparent number of protein-adsorbent interactions increases on clustered charge adsorbents, even when total adsorbent charge density is held constant (Table 3). The increased Z value for cytochrome c is consistent with better multivalent contact between adsorbent and adsorbate for proteins with inherent clusters when adsorbent charges are displayed in the form of clusters.

TABLE 3
Apparent average number of interacting groups (Z) for lysozyme
and cytochrome c adsorption on E and (E5) agarose in 10 mM
Tris, pH 8.0. The uncertainties that arise in determining the
hill dissociation constant, K_{d} from Hill plots were also incorporated
in calculating the errors in Z values.

| | Dispersed charge | Clustered charge | Ratio of Z |
|---|---|---|---|
| Protein | Adsorbent (E) | Adsorbent (E5) | values, E5/E |
| Lysozyme | 2.08 ± 0.02 | 1.86 ± 0.05 | 0.89 |
| Cytochrome c | 1.36 ± 0.07 | 2.15 ± 0.06 | 1.58 |

### EXAMPLE 5

Clustered Charge and Dispersed Charge Anion Exchange Adsorbents Using CNBr Activated Agarose

1 gm of CNBr-activated Sepharose 4B was washed with 1 mM HCl to remove additives added during lyophyllization. The gel swells to 3.5 mL. It was then suspended in 8 mL of binding buffer, 0.5 M NaHCO₃, pH 7.0 containing 0.5 M NaCl in a 15 mL Falcon tube. Lysinamide/pentalysinamide was dissolved in the same buffer and an aliquot to give desired ligand density was added to the gel suspension. For lysinamide 5 micromoles per mL of the gel and for pentalysinamide 1 micromole per mL of the gel was used. This was done to account for the contribution of 5 ε-amino groups per pentalysinamide molecule as against 1 ε-amino group per lysinamide molecule.

After ligand addition, the reaction tube was placed on a gyratory shaker at 4° C. overnight. Lower binding pH and reaction temperature suppress deprotonation of the -ε-amino group. This is important to restrict coupling of the ligands only via the α-amino groups. The suspension was then filtered and the supernatant analyzed for amino acid content by Ninhydrin assay and peptide content using Bicinchoninic acid assay. The gel was washed with 3× gel volume of binding buffer and then suspended in 12 mL of 0.1 M Tris-HCl buffer, pH 8 containing 0.5 M of NaCl, to quench the active sites still present. The tube is again placed on the gyratory shaker for 2 hrs. This is followed by filtering the suspension and washing the gel alternately with 0.1 M sodium acetate buffer, pH 5.0 containing 0.5 M NaCl (total wash volume was 5× of gel volume) and basic 0.1 M Tris-HCl buffer, pH 8.0 containing 0.5 M NaCl (total wash volume was 5× of gel volume). Finally the gel was suspended in 10 mM Tris-HCl buffer, pH 8.0, which is also the binding buffer for adsorption isotherms.

The initial candidate ligands were lysine and pentalysine for the dispersed and clustered charges adsorbents respectively, but these test ligands would form dipolar adsorbents. Lysine or pentalysine can be coupled to CNBr activated agarose via the α-amino group. This leaves the ε-amino and the α-carboxyl groups free to interact with the target molecule thereby creating a dipolar adsorbent. Such adsorbents have been used in RNA separation and are commercially available (25).

In this work, it is important that the adsorbents be purely cationic and not dipolar. So this limitation was bypassed by using the α-carboxyl blocked amide form of the amino acid and peptide. Furthermore, using the pKa calculation program SPARC, it was determined that in lysinamide the pKa of the α-amino group is 6.86 as compared to 8.90 in lysine. This allows for a low coupling pH of 7.0. At lower pH values, deprotonation of the ε-amino group in lysinamide is minimal and it is thus free to interact with the target molecule (26). The amino density for lysinamide-agarose and pentalysinamide-agarose was maintained constant at 5 mmole/mL gel by coupling 5 times more (monovalent) lysinamide than (pentavalent) pentalysinamide.

The coupling of an amino group to activated CNBr matrix results in a positively charged isourea linkage. So the lysinamide and pentalysinamide adsorbents have positive charge contribution due to the isourea linkage besides the positive charge contribution due to the ligand ε-amino groups. As the number of lysinamide molecules used to prepare the dispersed charges adsorbent is more than the number of pentalysinamide molecules used to prepare the clustered charges adsorbent (to maintain equivalent number of ε-amino groups), there are bound to be more positively charged isourea linkages in the dispersed charges adsorbent as compared to the clustered charges adsorbent. However, in spite of the higher number of positive charges in the dispersed charges adsorbent, better adsorbent performance was seen for the clustered charges adsorbent as discussed below.

### EXAMPLE 6

Adsorption Isotherms for Anionic Proteins

Adsorption isotherms for anionic proteins α-lactalbumin (calcium saturated and calcium depleted forms) and cytochrome b₅ (wild type and E11Q and E44Q mutant proteins) were prepared as described in Example 2.

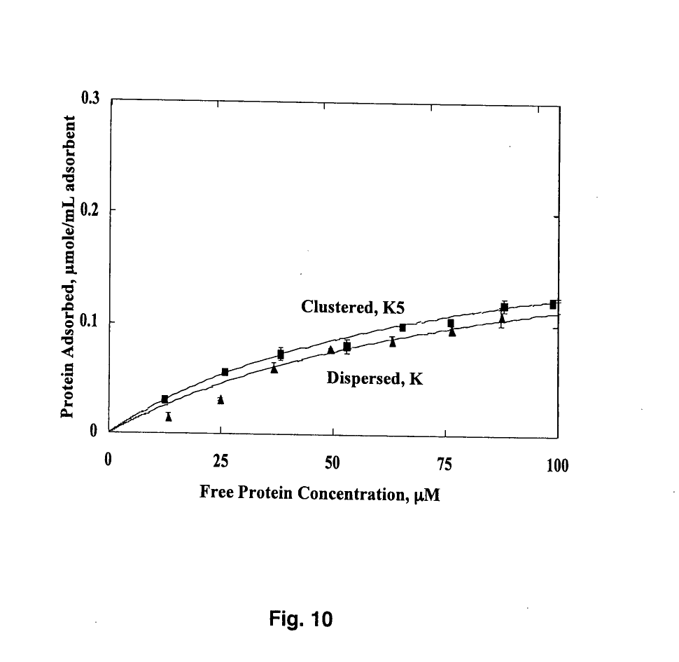

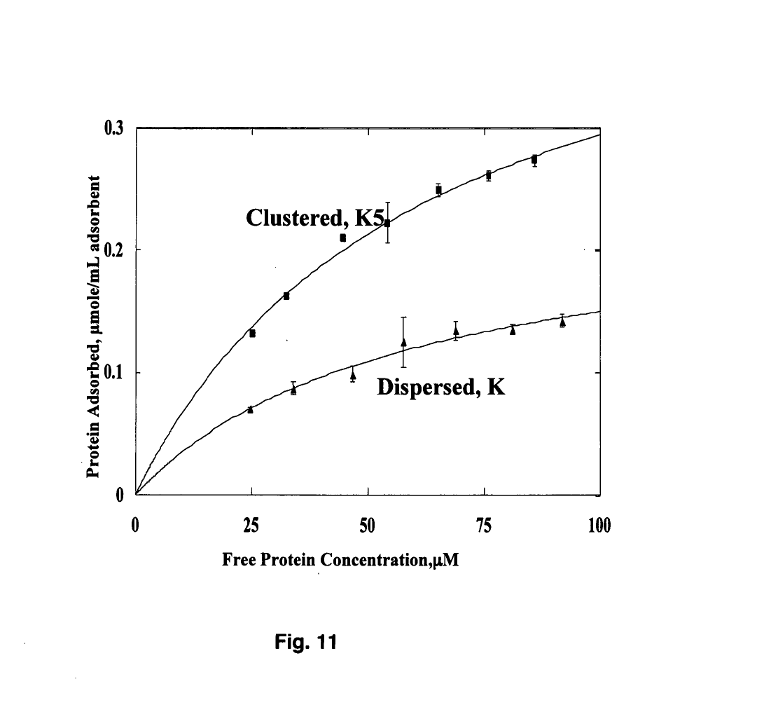

To compare the effect of random versus structured charge distribution, initially the Ca²⁺ saturated and Ca²⁺ depleted forms of the anionic protein, bovine α-lactalbumin, were adsorbed on lysinamide-agarose and pentalysinamide agarose. The amino group density was maintained at 5 μmole/mL adsorbent for both the adsorbents. From FIG. 10, it is clear that the Ca²⁺ saturated protein does not show increased binding on the clustered charges adsorbent as compared to the dispersed-charges adsorbent. The native protein has a very high affinity for calcium ion and the apparent dissociation constant for Ca²⁺ binding under physiological conditions is of the order of 10⁻⁷ M (27). Highly conserved Ca²⁺ binding aspartate residues are located at the junction between the two subdomains of the protein (28). The binding capacity for the Ca²⁺ saturated protein is lower than that for the Ca²⁺ depleted protein (FIGS. 10-11) because these aspartate residues are liganded to the Ca²⁺ ion.

In the Ca²⁺ depleted α-lactalbumin there is increased mobility and slightly higher solvent accessibility at the Ca²⁺ binding site as compared to when Ca²⁺ is present (29). Larger solvent accessibility is due to the charge repulsion between the carboxylates of the five aspartate residues in this region (29). This allows for better access of the aspartate cluster to the adsorbent. This may allow for multivalent contact with the clustered charge adsorbent and hence enhanced binding capacity as compared to the dispersed charge adsorbent. As shown in FIG. 11, for the Ca²⁺ depleted protein, there is a marked increase in the binding capacity from 0.22 μmole/mL adsorbent for the dispersed charges adsorbent to 0.48 μmole/mL adsorbent for the clustered charges adsorbent. This adequately demonstrates that the clustered charge anion exchanger recognizes the presence of an anionic cluster in the protein resulting in enhanced binding capacity as compared to when the same number of charges is displayed in a random array. When this cluster is not available due to liganding with Ca²⁺, this adsorbent behaves like the dispersed charge adsorbent.

In the second example, the anionic protein, recombinant rat microsomal cytochrome b₅ and its two mutant forms E11Q and E44Q were adsorbed on lysinamide-agarose and pentalysinamide-agarose in separate batch experiments. Earlier work involving single point mutations, demonstrated that cytochrome b₅ has a preferred ion exchange active cluster of glutamic acid residues (30,31). The mutation of Glu44 to Gln disrupts this cluster. The resultant mutant protein has a lower binding capacity for the anion exchanger MONO Q® from Amersham Biosciences as compared to the wild type cytochrome b₅. The mutant protein E11Q, on the other hand, did not exhibit a significant deviation in binding capacity as compared to the wild type protein indicating that Glu11 does not belong to any ion exchange active patch in the protein. As one Glu residue has been replaced by one Gln residue in each mutant, they are equivalent in terms of net charge at pH 8.0.

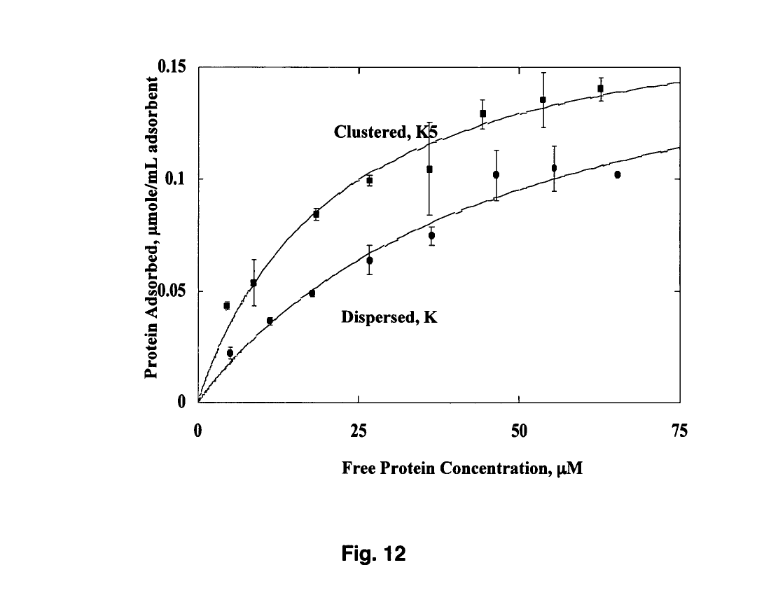

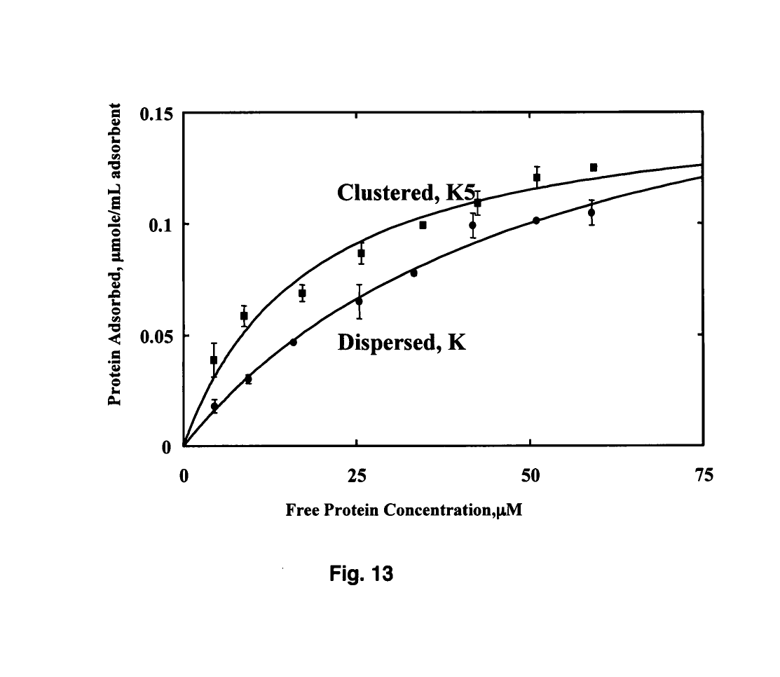

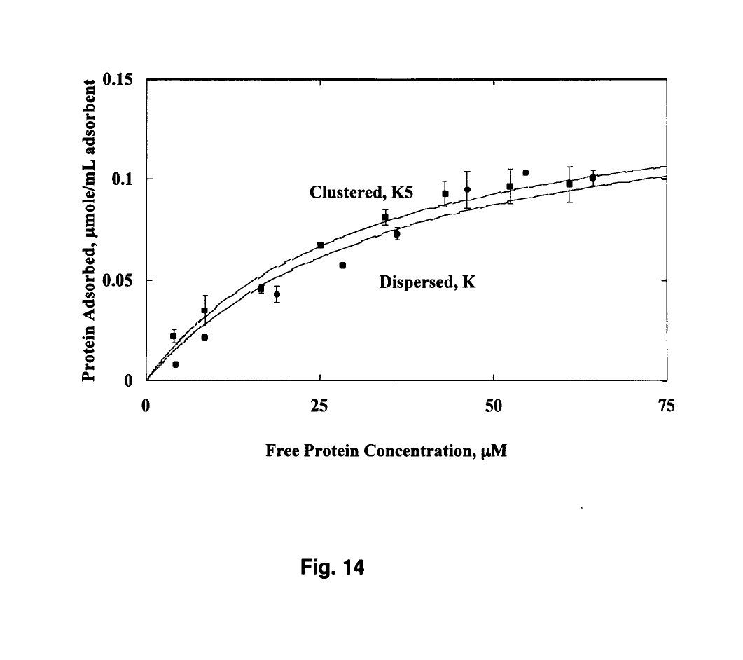

The adsorption isotherms for the wild type cytochrome b₅ (FIG. 12) shows an increase in binding capacity for the clustered charge adsorbent, as opposed to the dispersed-charges adsorbent. This protein has a cluster of Glu residues that probably allow for better binding with the clustered charges adsorbent. In the mutant E11Q, this ion exchange selective cluster is intact and as seen in FIG. 13, there is enhancement in its binding on the clustered charges adsorbent. However, there is a small drop in the overall binding capacity as compared to the wild type protein. This may be a result of lower negative charge density in the mutant due to loss of the Glu11 residue. For the mutant protein E44Q (FIG. 14) loss of the preferred ion exchange patch is evident as there is no improvement in the binding capacity or affinity for the clustered charge adsorbent as compared to the dispersed charge adsorbent. This proves that structuring of charges selectively increases binding affinity/capacity for target molecules. Minor charge related molecular changes are also recognized by implementing structuring of charges in ion exchange adsorbent design.

### EXAMPLE 7

Data Analysis

Protein adsorption data for the cation exchange adsorbents and cationic proteins at various salt concentrations were fit to the Langmuir-Freundlich equation:

$$Y = \frac{{Q}_{max} {X}^{{n}_{H}}}{{X}^{{n}_{H}} + {K}_{d}^{{n}_{H}}}$$

where Y is adsorbed protein concentration at equilibrium, X is free protein concentration at equilibrium, Qₘₐₓ is the maximum capacity of the adsorbent, n_{H} is the heterogeneity index and K_{d}ⁿ^{_{H}} is the dissociation constant.

Isotherms were fit to the data using non-linear Levenburg-Marquardt regression with LABfit (Universidade Federal de Campina Grande, Brazil) version 7.2.15. The initial guess vectors for the parameters were varied manually to ensure that a true global minimum was found. The mass balance (protein recovered in supernatant+protein eluted divided by protein originally added) for adsorption data was in the range 90-96%. Adsorption data fit well to a linear regression on Hill plots. The slope of the Hill plots gives the cooperativity factor, n_{H}, and the intercept gives the Hill binding coefficient, K. Using these K values, Z-plots were prepared for each protein-adsorbent combination and the apparent number of interacting sites, Z, was obtained from the slope.

Protein adsorption data for anion exchange adsorbents and anionic proteins were fit to the Langmuir equation. Isotherms were fit to the data using Igor Pro (WaveMetrics, Inc., Lake Oswego, Oreg., USA) version 4.04, which uses the Levenburg-Marquardt algorithm for parameter estimation. The initial guess vectors for the parameters were varied manually to ensure that a true global minimum was found. The mass balances (protein recovered in supernatant+protein eluted divided by protein originally added) for the adsorption data closed in the range 90-96%.

### EXAMPLE 8

Clustered Charge and Dispersed Charge Cation Exchange Adsorbents Using Aldehyde-Activated Agarose

As discussed in Example 5, coupling a ligand via an amino group to CNBr-activated agarose results in a positively charged ionic linkage. This positive character of the coupling linkage may neutralize the negative charge of glutamic acid. This effect, if present, would be more so for glutamic acid-agarose than pentaglutamic acid agarose. Thus, it is possible that enhancement in binding capacity with the clustered charges adsorbent could be because of the neutralization of charge for the dispersed charges adsorbent. To overrule this possibility E-agarose and E5-agarose were prepared by using aldehyde activated agarose. Aldehyde-activated agarose was acquired from Pierce Endogen, USA. Coupling via an amino group results in a secondary amine linkage which does not have ionic character. The adsorbents were prepared as per the manufacturer's protocol.

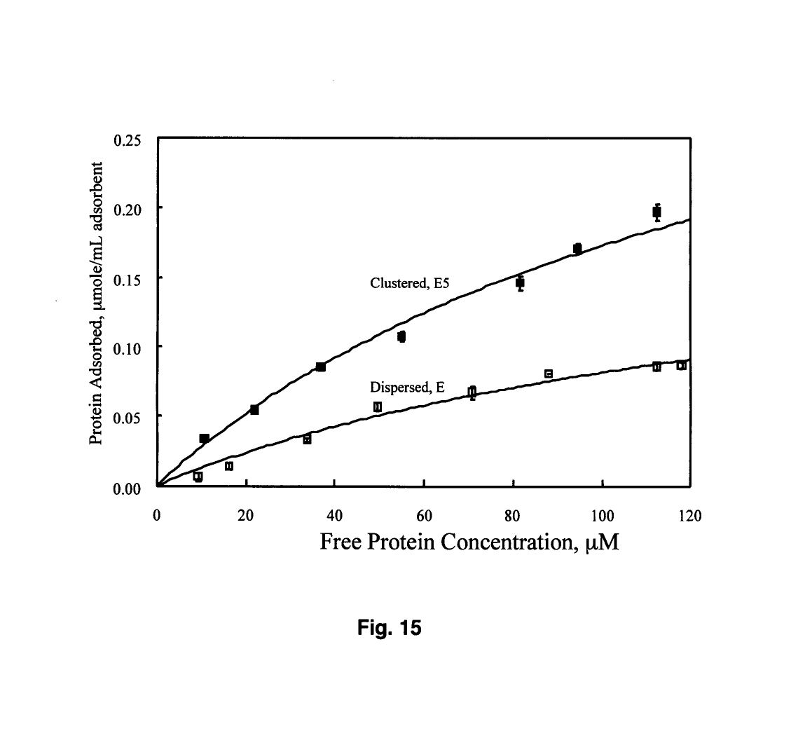

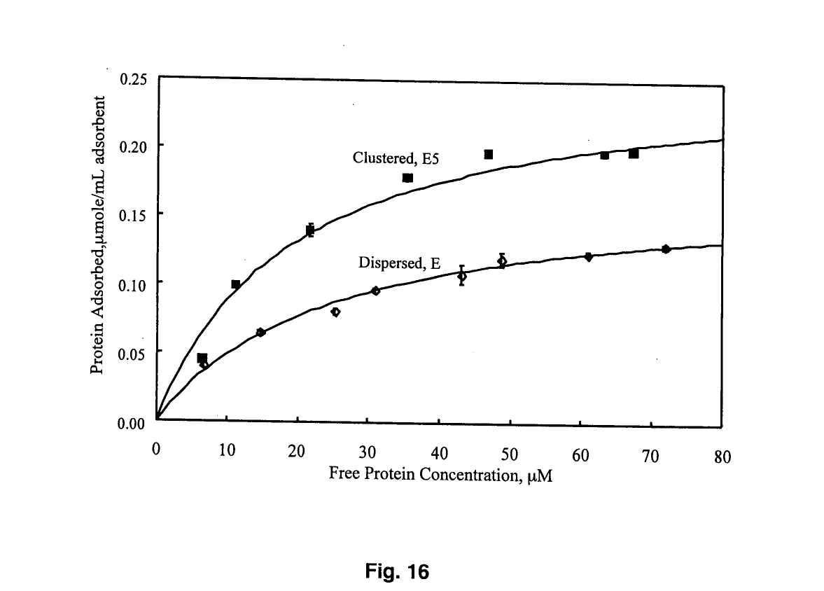

The adsorption isotherms for cytochrome c and lysozyme (FIGS. 3-4 and 15-16) with E-agarose and E5-agarose clearly show that similar results in terms of enhanced adsorbent capacity and selectivity were obtained for both CNBr-activated agarose and aldehyde-activated agarose when the charges are clustered on the matrix. This demonstrates that clustering of charges on a matrix can result in an adsorbent with high capacity and selectivity.

### EXAMPLE 9

Other Adsorbent Clusters

One skilled in the art can recognize that adsorbents with clustered ligands such as hydrophobic ligands or metal ligands can be prepared to improve adsorbent capacity and specificity for recovery of a number of molecules. It is contemplated that clusters of chelated metal ligands can be prepared which may be used to purify nucleic acids. Each nucleotide in a nucleic acid fragment contributes an aromatic base and thus the nucleic acid can be envisioned as a molecule with a cluster of aromatic bases which may selectively interact with a cluster of chelated metal ligands. Thus the compositions and method presented herein may be used to purify different nucleic acids based on their configuration such as single stranded RNA, structured double stranded RNA, linear double stranded DNA or closed circular double stranded plasmid DNA. These different configurations will potentially display a different array of aromatic base clusters which will help to selectively purify a nucleic acid with a specific configuration from a milieu that contains two or more fragments of different configurations using an adsorbent which displays small clusters of chelated metal.

It is contemplated that a clustered metal adsorbent may be prepared by coupling pentacysteine to CNBr activated sepahrose as described in Example 1. Then the SH group of cysteine can be crosslinked using a heterobifunctional crosslinker such as Sulpfo-GMBS (Pierce Endogen crosslinking agent) to the amino group of iminodiacetic acid derivative of lysine. This will give an adsorbent with five iminodiacetic acid molecules closely associated in a cluster which can then be charged with a transition metal of choice to form clusters of chelated metal ligand. Nucleic acid that selectively binds this adsorbent may be eluted with imidazole.

The following references are cited herein.
1. Bibak, et al. Anal. Biochem. 333(1):57-64 (2004).
2. Hu, et al. Biotechnol. Appl. Biochem. 40:89-94 (2004).
3. Mueller, et al. U.S. Pat. No. 6,149,994, (2000).
4. Hendriks, et al. U.S. Pat. No. 6,617,443, (2003).
5. Jianrong, et al. J. Chromatogr., 691, 263-271 (1995).
6. Muranaka, K., & Tsuda, T. U.S. Pat. No. 6,689,820, (2004).
7. Etheve, et al. Colloids Surf., B, 28(4):285 (2003).
8. Schwarz, R. M., & Dayhoff, M. O. Science, 199:395-403 (1978).
9. Diamond, et al. J. Biol. Chem., 266(33):22761-22769 (1991).
10. Brendel, V., & Karlin, S. Proc. Natl. Acad. Sci. U.S.A., 86:5698-5702 (1989).
11. Evans, R. M. Science, 240:889-895 (1988).
13. Chicz, R., & Regnier, F. E. J. Chromatogr., 443:193-203 (1988).
14. Chicz, R., & Regnier, F. E. Anal. Chem., 61(18):2059-2066 (1989).
15. Heimenz, P. C. Principles of Colloid and Surface Chemistry. Second ed. Dekker, NY, 1986.
16. Shin, et al. Biotechnol. Prog., 19(3):928-935 (2003).
17. Zeng, X., & Ruckenstein, E. J. Membr. Sci., 148(2):195-205 (1998).
18. Dickerson, et al. J. Biol. Chem., 246(5):1511-1535 (1971).
19. Capaldi, R. A. (1990) Structure and function of cytochrome c oxidase. Annu. Rev. Biochem., 59:569-596 (1990).
20. Jennissen, H. P. Int. J. Bio-Chromatogr., 5(2):131-163 (2000).
21. Gill, et al. J. Colloid Interface Sci., 167:1-7 (1994).
22. Boardman, N. K., & Partridge, S. M. (Biochem. J., 59:543-552 (1955).
23. Velayudhan, A. & Horvath, C. S. J. Chromatogr., 367:160-162 (1987).
24. Whitely, et al. J. Chromatogr. 465:137-156 (1989).
25. Jones, et al. Nucleic Acids Res., 3(6):1569-1576 (1976).
26. Alkjaersig, et al. J. Biol. Chem. 234:832-837 (1959).
27. Stuart, et al. Nature, 324:84-87 (1986).
28. Chrysina, et al. J. Biol. Chem., 275:37021-37029 (2000).
29. Kronman, M. J. Crit. Rev. Biochem. Mol. Biol. 24:565-667 (1989).
30. Gill, et al. J. Chromatogr., 684(1):55-63 (1994).
31. Roush, et al. J. Chromatogr., 704(2):33949 (1995).

Any publications mentioned in this specification are indicative of the levels of those skilled in the art to which the invention pertains. Further, these publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An adsorbent composition comprising;
a support material; and
a ligand associated with said support material, said ligand comprising a cluster of functional groups such that the majority of the clusters contain substantially the same number of functional groups.

2. The composition of claim 1, wherein the number of said functional groups in each cluster is about 3 to about 10.

3. The composition of claim 1, wherein said functional groups comprise carboxylate moieties, amine moieties, or chelating moieties.

4. The composition of claim 1, wherein said functional groups comprise a sulfopropyl group, a diethylaminoethyl group, a sulfhydryl group or a carboxymethyl group.

5. The composition of claim 1, where said ligands comprise amino acids, iminodiacetic acid, transition metal ions, nitrilotriacetic acid, nucleotides, sugars, or fatty acids.

6. The composition of claim 1, wherein said support material comprises agarose, alginate, polystyrene, cellulose, polyacrylamide, dextran, polyethylene glycol, fullerenes, glass, graphite or a combination thereof.

7. The composition of claim 1, wherein said support material is porous, magnetic or weighted for rapid settling.

8. The composition of claim 1, wherein said support material comprises a gel, a glass slide, one or more fibers, a microchip, a paper or beads.

9. A method for adsorbing a biomolecule onto an adsorbent composition comprising:
contacting said biomolecule with the adsorbent composition of claim 1.

10. The method of claim 9, wherein adsorption of said biomolecule is either for qualitative analysis or for bulk recovery of said biomolecule.

11. The method of claim 9, wherein said method is carried out in a mode comprising fast liquid chromatography, expanded bed chromatography, simulated bed chromatography, electrophoresis or capillary electrophoresis.

12. The method of claim 9, wherein said biomolecule is a protein, a nucleic acid, an oligonucleotide, a virus, or a cell organelle.

13. A kit comprising the adsorbent composition of claim 1.

14. The kit of claim 13, further comprising a column, a glass slide, a microchip, buffers or a combination thereof.

15. The kit of claim 13, wherein said adsorbent composition is useful for qualitative analysis or recovery and purification of a biomolecule.

16. An adsorbent composition comprising:
a support material; and
a peptide ligand associated with said support material, wherein said peptide comprising about 3 to about 10 amino acids or derivatives thereof.

17. The composition of claim 16, wherein said peptide comprises glutamic acid.

18. The composition of claim 16, wherein said peptide comprises lysinamide.

19. The composition of claim 16, wherein said peptide comprises cysteine and said ligand further comprises iminodiacetic acid.

20. The composition of claim 16, wherein said support material comprises agarose.
